(19) United States
(12) Patent Application Publication
(10) Pub. No.: US 2009/0069361 A1
(43) Pub. Date: Mar. 12, 2009

(54) METHODS OF TREATMENT USING (+)- ISOPROPYL 2-METHOXYETHYL 4-(2-CHLORO-3-CYANO-PHENYL)-1, 4-DIHYDRO-2, 6-DIMETHYL-PYRIDINE-3, 5-DICARBOXYLATE

(75) Inventors: Axel Unterbeck, Fort Lee, NJ (US); Michael De Vivo, New York, NY (US); Gregory M. Rose, Guilford, CT (US)

Correspondence Address: DARBY & DARBY P.C., P.O. BOX 770, Church Street Station, New York, NY, 10008-0770, US

(73) Assignee: Memory Pharmaceuticals Corporation, Montvale, NJ (US)

(21) Appl. No.: 12/261,649

(22) Filed: Oct. 30, 2008

Related U.S. Application Data
(63) Continuation of application No. 10/580,008, filed on May 4, 2007, now abandoned.
(60) Provisional application No. 60/523,664, filed on Nov. 21, 2003.
(60) Provisional application No. 60/608,116, filed on Sep. 9, 2004.

(51) Int. Cl. A61K 31/435

(52) U.S. Cl. 514/277

(57) ABSTRACT

The present invention relates to methods of treatment using the compound (+)-isopropyl 2-methoxyethyl 4-(2-chloro-3-cyano-phenyl)-1,4-dihydro-2,6-dimethyl-pyridine-3,5-dicarboxylate, as a sole active agent or in combination with other pharmacological agents.

This application claims the benefit of Ser. No. 60/523,664, filed Nov. 21, 2003, and Ser. No. 60/608,116, filed Sep. 9, 2004.

The present invention relates to methods of treatment using the compound (+)-isopropyl 2-methoxyethyl, 4-(2-chloro-3-cyano-phenyl)-1,4-dihydro-2,6-dimethyl-pyridine-3,5-dicarboxylate, as a sole active agent or in combination with other pharmacological agents.

### BACKGROUND OF THE INVENTION

Meier at al. (U.S. Pat. No. 5,665,740), the entire disclosure of which is hereby incorporated by reference, disclose that the compound, (+)-isopropyl 2-methoxyethyl 4-(2-chloro-3-cyano-phenyl)-1,4-dihydro-2,6-dimethyl-pyridine-3,5-dicarboxylate, has a positive effect on learning and memory powers and has antidepressant potential.

The condition of memory impairment is manifested by impairment of the ability to learn new information and/or the inability to recall previously learned information. Memory impairment is a primary symptom of dementia and can also be a symptom associated with a variety of diseases and conditions such as Alzheimer's disease or age-related cognitive decline.

The present invention relates to further uses of (+)-isopropyl 2-methoxyethyl 4-(2-chloro-3-cyano-phenyl)-1,4-dihydro-2,6-dimethyl-pyridine-3,5-dicarboxylate based on the useful spectrum of pharmacological activities that this compound exhibits, particularly with regard to treatments for memory and/or cognitive impairment.

### SUMMARY OF THE INVENTION

The present invention includes methods of treating patients, especially humans, suffering from Mild Cognitive Impairment (MCI), comprising administering an effective amount of (+)-isopropyl 2-methoxyethyl 4-(2-chloro-3-cyano-phenyl)-1,4-dihydro-2,6-dimethyl-pyridine-3,5-dicarboxylate. MCI is a condition characterized by mild recent memory loss without dementia or significant impairment of other cognitive functions, such as orientation, language, and attention. Characteristics of MCI include memory complaint and abnormal memory for age, however with normal activities of daily living, normal general cognitive functioning, and no dementia.

The compound can also be used in methods of treating patients, especially humans, suffering from neuronal damage as a result of CNS hypoxia, for instance as a result of Coronary Artery Bypass Grafting (CABG), and perinatal hypoxia, especially in the treatment of memory impairment and/or cognitive impairment due to such neuronal damage, comprising administering an effective amount of (+)-isopropyl 2-methoxyethyl 4-(2-chloro-3-cyano-phenyl)-1,4-dihydro-2,6-dimethyl-pyridine-3,5-dicarboxylate.

The present invention includes methods of treating patients, especially humans, suffering from memory impairment and/or cognitive impairment due to, for example, schizophrenia, Huntington's disease, Pick's disease, Creutzfeld-Jakob disease, and other neurological conditions, comprising administering an effective amount of (+)-isopropyl 2-methoxyethyl 4-(2-chloro-3-cyano-phenyl)-1,4-dihydro-2,6-dimethyl-pyridine-3,5-dicarboxylate.

The present invention also includes methods for treating patients, especially humans, suffering from multiple sclerosis, especially with regard to memory and/or cognitive impairment as a result thereof, comprising administering an effective amount of (+)-isopropyl 2-methoxyethyl 4-(2-chloro-3-cyano-phenyl)-1,4-dihydro-2,6-dimethyl-pyridine-3,5-dicarboxylate.

In accordance with another embodiment of the invention there is provided a method of treating a patient, especially a human, suffering from epilepsy-related memory and/or cognitive impairment comprising administering to the patient an effective amount of (+)-isopropyl 2-methoxyethyl 4-(2-chloro-3-cyano-phenyl)-1,4-dihydro-2,6-dimethyl-pyridine-3,5-dicarboxylate.

Additionally, in accordance with the invention, there is provided a method of treating a patient, especially a human, suffering from conditions of memory and/or cognition impairment due to disease states including, for example, attention deficit disorder (ADD) and attention deficit hyperactivity disorder (ADHD), and the like, comprising administering to the patient an effective amount of (+)-isopropyl 2-methoxyethyl 4-(2-chloro-3-cyano-phenyl)-1,4-dihydro-2,6-dimethyl-pyridine-3,5-dicarboxylate.

In accordance with a further aspect of the invention there is provided a method of treating a patient, especially a human, suffering from tinnitis and/or other symptoms of cerebral insufficiency, comprising administering to the patient an effective amount of (+)-isopropyl 2-methoxyethyl 4-(2-chloro-3-cyano-phenyl)-1,4-dihydro-2,6-dimethyl-pyridine-3,5-dicarboxylate.

One of ordinary skill in the art will recognize that the compound, isopropyl 2-methoxyethyl 4-(2-chloro-3-cyano-phenyl)-1,4-dihydro-2,6-dimethylpyridine-3,5-dicarboxylate, possesses an asymmetric carbon atom and thus is capable of existing in the form of optical isomers, as well as in the form of racemic or nonracemic mixtures thereof. All of these compounds, including racemates, nonracemic mixtures of enantiomers, substantially pure, and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of the invention can likewise be obtained by utilizing optically active starting materials in chiral syntheses processes under reaction conditions that do not cause racemization.

The compounds can be administered as the sole active agent or in combination with other pharmaceutical agents such as other agents used in the treatment of cognitive impairment and/or memory loss, e.g., nicotinic −7 agonists, PDE4 inhibitors, calcium channel blockers (e.g., amlodipine, felodipine, isradipine, lacidipine, lercanidipine, nicardipine, nifedipine, nimodipine, nitrendipine, and nisoldipine), muscarinic ml and m2 modulators, adenosine receptor modulators, ampaidnes, NMDA-R modulators (e.g., memantine (Namenda®), mGluR modulators, dopamine modulators, serotonin modulators, and canabinoid modulators. In such combinations, each active ingredient can be administered either in accordance with their usual dosage range or a dose below their usual dosage range.

For example, the invention includes methods for treating memory and/or cognitive impairment associated with Alzheimers disease comprising administering to a patient (e.g., a human), simultaneously or sequentially, the compound of the invention and another agent used in the treatment of Alzheimers disease selected from Akatinol, Neotropin, Eldepryl, Estrogen, and Clioquinol. In methods using simultaneous administration, the agents can be present in a combined composition or can be administered separately.

The invention also includes methods for treating memory and/or cognitive impairment associated with schizophrenia comprising administering to a patient (e.g., a human), simultaneously or sequentially, the compound of the invention and another agent used in the treatment of schizophrenia such as Clozaril, Zyprexa, Risperidone, and Seroquel. In methods using simultaneous administration, the agents can be present in a combined composition or can be administered separately.

The invention also includes methods for treating memory and/or cognitive impairment associated with Parkinson's disease comprising administering to a patient (e.g., a human), simultaneously or sequentially, the compound of the invention and another agent used in the treatment of Parkinson's disease such as Levodopa, Parlodel, Permax, Mirapex, Tasmar, Comtan, Kemadrin, Artane, and Cogentin. In methods using simultaneous administration, the agents can be present in a combined composition or can be administered separately.

In addition, the invention includes methods for treating memory and/or cognitive impairment associated with Huntington's disease comprising administering to a patient(e.g., a human), simultaneously or sequentially, the compound of the invention and another agent used in the treatment of Huntington's disease such as Amitriptyline, Imipramine, Despiramine, Nortriptyline, Paroxetine, Fluoxetine, Sertraline, Tetrabenazine, Haloperidol, Chlorpromazine, Thioridazine, Sulpride, Quetiapine, Clozapine, and Risperidone. In methods using simultaneous administration, the agents can be present in a combined composition or can be administered separately.

Further, the invention includes methods for treating memory and/or cognitive impairment associated with Attention Deficit Hyperactivity Disorder (ADHD) comprising administering to a patient (e.g., a human), simultaneously or sequentially, the compound of the invention and another agent used in the treatment of ADHD such as Ritalin, Dexedrine, Dextrostat, Cylert, and Adderall. In methods using simultaneous administration, the agents can be present in a combined composition or can be administered separately.

The invention also includes methods for treating memory and/or cognitive impairment associated with depression comprising administering to a patient (e.g., a human), simultaneously or sequentially, the compound of the invention and another agent used in the treatment of depression such as Prozac, Zoloft, Paxil, Reboxetine, Wellbutrin, Olanzapine, Fluoxetine, Elavil, Tofranil, Pamelor, Nardil, Parnate, Desyrel, Effexor, Desyrel, Vivactil, Sinequan, Pamate, Zyprexa, Tryptanol, Serzone, Risperidal, Haldol, Faverin, Seroxat, Remeron, and Nortrilene. In methods using simultaneous administration, the agents can be present in a combined composition or can be administered separately.

Also included within the invention are methods for treating memory and/or cognitive impairment associated with dementia comprising administering to a patient (e.g., a human), simultaneously or sequentially, the compound of the invention and another agent used in the treatment of dementia selected from Thioridazine, Haloperidal, and Risperidone. In methods using simultaneous administration, the agents can be present in a combined composition or can be administered separately.

The invention also includes methods for treating memory and/or cognitive impairment associated with epilepsy comprising administering to a patient (e.g., a human), simultaneously or sequentially, the compound of the invention and another agent used in the treatment of epilepsy such as Dilantin, Luminol, Tegretol, Depakote, Depakene, Zarontin, Neurontin, Barbita, Solfeton, and Felbatol. In methods using simultaneous administration, the agents can be present in a combined composition or can be administered separately.

In addition, the invention includes methods for treating memory and/or cognitive impairment associated with bipolar disorder comprising administering to a patient (e.g., a human), simultaneously or sequentially, the compound of the invention and another agent used in the treatment of bipolar disorder such as Lithium, Zyprexa, Depakote, and Zyprexa. In methods using simultaneous administration, the agents can be present in a combined composition or can be administered separately.

Furthermore, the invention includes methods for treating memory and/or cognitive impairment associated with multiple sclerosis comprising administering to a patient (e.g., a human), simultaneously or sequentially, the compound of the invention and another agent used in the treatment of multiple sclerosis such as Detrol, Ditropan XL, OxyContin, Betaseron, Avonex, Azothioprine, Methotrexate, and Copaxone. In methods using simultaneous administration, the agents can be present in a combined composition or can be administered separately.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacolinetic profile of the compound, and the presence of any deleterious side-effects, among other considerations.

The compound of the invention can be administered alone or as an active ingredient of a formulation. Thus, the present invention also includes pharmaceutical compositions of the compound of the invention, containing, for example, one or more pharmaceutically acceptable carriers, and/or one or more active agents.

In view of their affinity to L-type calcium channels, the compound of the present invention can be administered to anyone requiring blocking of L-type calcium channels. Administration may be accomplished according to patient needs, for example, orally, nasally, parenterally (subcutaneously, intraveneously, intramuscularly, intrastemally and by infusion), rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The active compound of the invention should be present in these preparations in a concentration of 0.1 to 99.5% by weight, preferably of 0.5 to 95% by weight of the total mixture. In general, it has proven advantageous to administer the active compound of the invention in total amounts of about 0.01 to about 50 mg/kg, preferably in total amounts of about 0.1 mg/kg to 10 mg/kg of body weight every 24 hours, if appropriate in the form of several individual doses, to achieve the desired result.

The racemic compounds can be synthesized by various procedures, for example, as described in U.S. Pat. No. 5,665,740. For example, 2-chloro-3-cyanobenzaldehyde can be reacted with 2-methoxyethyl acetoacetate to obtain 2-methoxyethyl 2-acetyl-3-(2-chloro-3-cyano)-2-propenoate. This compound is then further reacted with isopropyl amino-2-butenoate to obtain racemic isopropyl 2-methoxyethyl 4-(2-chloro-3-cyano-phenyl)-1,4-dihydro-2,6-dimethyl-pyridine-3,5-dicarboxylate. (See Examples I and 1 of U.S. Pat. No. 5,665,740.) (+)-isopropyl 2-methoxyethyl 4-(2-chloro-3-cyano-phenyl)-1,4-dihydro-2,6-dimethyl-pyridine-3,5-dicarboxylate can be obtained by subjecting the racemate to chiral chromatography. (See Example 2 of U.S. Pat. No. 5,665,740.)

The optical isomer can also be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compound of the invention can likewise be obtained by utilizing optically active starting materials in chiral syntheses processes under reaction conditions that do not cause racemization.

In addition, due to their affinity to L-type calcium channels, labeled derivatives of the compounds of the invention (e.g., C¹¹ or F¹⁸ labeled derivatives), can be used in neuroimaging of the receptors within, e.g., the brain. Thus, using such labeled agents in vivo imaging of the receptors can be performed using, e.g., PET imaging.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made by the procedures described in U.S. Pat. Nos. 5,846,514 and 6,334,997, both of which are hereby incorporated by reference. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The synthesis of radiolabeled compounds VIA organometallic intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem (1981), 64(1-2), 9-32. CODEN: JRACBN ISSN:0022-4081, CAN 95:76229 AN 1981:476229 CAPLUS, each of which is hereby incorporated by reference.

In the foregoing, all temperatures are set forth uncorrected in degrees Celsius; and, unless otherwise indicated, all parts and percentages are by weight.

From the foregoing description, one skilled in the art can easily ascertain the essential characteristics of this invention and, without departing from the spirit and scope thereof, can make various changes and modifications of the invention to adapt it to various usages and conditions.

We claim:

1. A method of treating a patient suffering from (a) Mild Cognitive Impairment, (b) neuronal damage as a result of CNS hypoxia, (c) memory impairment and/or cognitive impairment due to schizophrenia, Huntington's disease, Pick's disease, Creutzfeldt-Jakob disease, or other neurological conditions, (d) memory and/or cognition impairment due to disease states selected from attention deficit disorder (ADD), attention deficit hyperactivity disorder (ADHD), and multiple sclerosis, (e) epilepsy-related memory and/or cognitive impairment, or (f) tinnitus and/or other symptoms of cerebral insufficiency comprising administering to the patient an effective amount of (+)-isopropyl 2-methoxyethyl 4-(2-chloro-3-cyano-phenyl)-1,4-dihydro-2,6-dimethyl-pyridine-3,5-dicarboxylate.

2. A method according to claim 1, wherein the patient is suffering from neuronal damage that is the result of Coronary Artery Bypass Grafting (CABG) or perinatal hypoxia.

3. A method according to claim 1, wherein the patient is suffering from neuronal damage and said patient is treated for memory impairment and/or cognitive impairment due to such neuronal damage.

4. A method according claim 1 further comprising administering to said patient one or more further pharmaceutical agents for the treatment of cognitive impairment and/or memory loss, wherein said one or more further pharmaceutical agents are selected from nicotinic α-7 agonists, PDE4 inhibitors, calcium channel blockers, muscarinic m1 and m2 modulators, adenosine receptor modulators, ampakines, NMDA-R modulators, mGluR modulators, dopamine modulators, serotonin modulators, and canabinoid modulators.

5. A method according to claim 4, wherein said one or more further pharmaceutical agents are selected from amlodipine, felodipine, isradipine, lacidipine, lercanidipine, nicardipine, nifedipine, nimodipine, nitrendipine, nisoldipine, and memantine.

6. A method of treating a patient suffering from memory and/or cognitive impairment associated with schizophrenia according to claim 1, further comprising administering to said patient an effective amount of one or more further pharmaceutical agents selected from Clozaril, Zyprexa, Risperidone, and Seroquel.

7. A method of treating a patient suffering from memory and/or cognitive impairment associated with Huntington's disease according to claim 1, further comprising administering to said patient an effective amount of one or more further pharmaceutical agents selected from Amitriptyline, Imipramine, Despiramine, Nortriptyline, Paroxetine, Fluoxetine, Sertraline, Tetrabenazine, Haloperidol, Chlorpromazine, Thioridazine, Sulpride, Quetiapine, Clozapine, and Risperidone.

8. A method of treating a patient suffering from memory and/or cognitive impairment associated with Attention Deficit Hyperactivity Disorder (ADHD) according to claim 1, further comprising administering to said patient an effective amount of one or more further pharmaceutical agents selected from Ritalin, Dexedrine, Dextrostat, Cylert, and Adderall.

9. A method of treating a patient suffering from memory and/or cognitive impairment due to multiple sclerosis according to claim 1, further comprising administering to said patient an effective amount of one or more further pharmaceutical agents selected from Detrol, Ditropan XL, OxyContin, Betaseron, Avonex, Azothioprine, Methotrexate, and Copaxone.

10. A method of treating a patient suffering from epilepsy-related memory and/or cognitive impairment according to claim 1, further comprising administering to said patient an effective amount of one or more further pharmaceutical agents selected from Dilantin, Luminol, Tegretol, Depakote, Depakene, Zarontin, Neurontin, Barbita, Solfeton, or Felbatol.

11. A method of treating a patient suffering from memory and/or cognitive impairment associated with Alzheimer's disease, Parkinson's disease, depression, dementia, or bipolar disorder comprising administering to the patient an effective amount of (+)-isopropyl 2-methoxyethyl 4-(2-chloro-3-cyano-phenyl)-1,4-dihydro-2,6-dimethyl-pyridine-3,5-dicarboxylated and an effective amount of one or more further pharmaceutical agents.

12. The method of claim 11, wherein the patient is suffering from memory and/or cognitive impairment associated with Alzheimer's disease and the one or more further pharmaceutical agents is selected from Akatinol, Neotropin, Eldepryl, Estrogen, and Clioquinol.

13. The method of claim 11, wherein the patient is suffering from memory and/or cognitive impairment associated with Parkinson's disease and the one or more further pharmaceutical agents is selected from Levodopa, Parlodel, Permax, Mirapex, Tasmar, Comtan, Kemadrin, Artane, and Cogentin.

14. The method of claim 11, wherein the patient is suffering from memory and/or cognitive impairment associated with depression and the one or more further pharmaceutical agents is selected from Prozac, Zoloft, Paxil, Reboxetine, Wellbutrin, Olanzapine, Fluoxetine, Elavil, Tofranil, Pamelor, Nardil, Pamate, Desyrel, Effexor, Vivactil, Sinequan, Parnate, Zyprexa, Tryptanol, Serzone, Risperidal, Haldol, Faverin, Seroxat, Remeron, and Nortrilene.

15. The method of claim 11, wherein the patient is suffering from memory and/or cognitive impairment associated with dementia and the one or more further pharmaceutical agents is selected from Thioridazine, Haloperidal, and Risperidone.

16. The method of claim 11, wherein the patient is suffering from memory and/or cognitive impairment associated with bipolar disorder and the one or more additional pharmaceutical agents is selected from Lithium, Depakote, and Zyprexa.

17. A method according to claim 1 wherein said patient is human.

18. A pharmaceutical composition comprising (+)-isopropyl 2-methoxyethyl 4-(2-chloro-3-cyano-phenyl)-1,4-dihydro-2,6-dimethyl-pyridine-3,5-dicarboxylate and one or more additional pharmaceutical agents selected from Lithium, Depakote, and Zyprexa.

19. A pharmaceutical composition according to claim 16, further comprising one or more further pharmaceutical agents selected from nicotinic α-7 agonists, PDE4 inhibitors, calcium channel blockers, muscarinic m1 and m2 modulators, adenosine receptor modulators, ampakines, NMDA-R modulators, mGluR modulators, dopamine modulators, serotonin modulators, and canabinoid modulators.
